(19) United States
(12) Patent Application Publication
(10) Pub. No.: US 2006/0134624 A1
(43) Pub. Date: Jun. 22, 2006

(54) Polymerase-based protocols for the introductions of deletions and insertions

(76) Inventor: John C. Salerno, Averill Park, NY (US)

Correspondence Address: ELMORE PATENT LAW GROUP, PC, 209 MAIN STREET, N. CHELMSFORD, MA, 01863, US

(21) Appl. No.: 10/544,434

(22) PCT Filed: Feb. 6, 2004
(86) PCT No.: PCT/US04/03497

§ 371 Date: Jan. 17, 2006

Related U.S. Application Data
(60) Provisional application No. 60/445,689, filed on Feb. 6, 2003.
(60) Provisional application No. 60/445,703, filed on Feb. 6, 2003.
(60) Provisional application No. 60/446,045, filed on Feb. 6, 2003.
(60) Provisional application No. 60/445,704, filed on Feb. 6, 2003.
(60) Provisional application No. 60/474,063, filed on May 29, 2003.

(51) Int. Cl. C12Q 1/68; C12P 19/34; C12N 15/87

(52) U.S. Cl. 435/6; 435/455; 435/91.2

(57) ABSTRACT

The invention relates to primers, libraries of primer, kits and methods for site-specific in vitro mutagenesis comprising: (a) cloning a parental polynucleotide into a vector comprising a cloning site, thereby obtaining a cloned product; (b) denaturing the cloned product, thereby obtaining a single-stranded polynucleotide template; (c) hybridizing at least one mutagenized oligonucleotide primer to the single-stranded polynucleotide template, thereby obtaining a first heteroduplex; (d) subjecting the first heteroduplex to linear amplification, thereby obtaining amplified products; (e) reacting the amplified products with a ligase, thereby obtaining ligated products; (f) denaturing the ligated products, thereby obtaining single stranded mutated polynucleotides; (g) hybridizing the single stranded mutated polynucleotides with a second oligonucleotide primer thereby obtaining second hybridized complexes; (h) copying the second hybridized complex and ligating the double stranded product thereof; thereby obtaining a circular double stranded mutated polynucleotide; (i) transforming the double-stranded mutated polynucleotide into a bacterial host, thereby obtaining transformants.

### RELATED APPLICATIONS

This application claims the benefit of U.S. Provisional Application No. 60/445,689, filed on Feb. 6, 2003, U.S. Provisional Application No. 60/445,703, filed on Feb. 6, 2003, U.S. Provisional Application No. 60/446,045, filed on Feb. 6, 2003, U.S. Provisional Application No. 60/445,704, filed on Feb. 6, 2003 and U.S. Provisional Application No. 60/474,063, filed on May 29, 2003, Docket No. RPI-812, entitled “Parental Suppression via Polymerase-based Protocols for the Introduction of Deletions and Insertions.” The entire teachings of the above applications are incorporated herein by reference.

### BACKGROUND OF THE INVENTION

In recent years a number of methods have come into common use that allow the generation of site directed mutants without subcloning based on polymerase activity. This technology is mature enough to allow the sale of a number of mutagenesis kits that are capable of producing point mutants and in some case insertion and deletion mutants (‘indels’).

An important class of polymerase-based mutagenesis methods use two complementary or partially complementary primers together with a thermostable polymerase to produce linearly amplified, double stranded linear DNA. The amplification is linear because primers binding to linear products face the wrong way (3′ out) to serve as primers for elongation. Although these methods are powerful, they contain flaws that limit their application and require expensive and delicate ‘ultracompetent’ cells for transformation because the products are linear.

A second class of mutagenesis methods use a T4 polymerase and a T4 ligase to make a single mutant copy which forms part of a hybrid circular duplex with the parental template from which it was copied. A second forward selection primer is included allowing partial suppression of parentals based on repair of an antibiotic resistance gene or suppression of a restriction site. The production of circular duplex DNA is highly desirable, but the hybrid nature of the duplex DNA limits the selection to 50% unless additional rounds plasmid preparation and transformation are included. This is so cumbersome that it is generally easier to sequence extra colonies. In addition, the single cycle limits the production of mutant DNA.

However, a need exists to further improve the efficiency of these methods.

### SUMMARY OF THE INVENTION

INSULT, a novel method for the creation of insertions, deletions, and point mutations without subcloning, requires only one new primer per mutant, and produces circular plasmids, obviating the need for special ‘ultracompetent’ cells. The method includes cycles of linear amplification with a thermophilic polymerase, and nick repair after each cycle with a thermophilic ligase. After production of multiple single stranded copies of circular mutation bearing plasmid DNA, addition of a ‘generic’ primer followed by one or more polymerase reaction cycles generates double stranded circular DNA bearing the desired mutation.

The present inventions relate to a set of methods which allow the production of site directed mutants via a novel polymerase based strategy which combines the strengths of both of the older methods. The results are high yields of mutant DNA, closed circular double stranded products which obviate the need for specialized ‘ultracompetent’ cells, and protocols which require only one new primer per mutant.

The invention relates to kits and methods for site-specific in vitro mutagenesis or combinatorial mutagenesis comprising:
(a) cloning a parental polynucleotide (such as polynucleotide comprising a coding sequence or gene) into a vector comprising a cloning site, thereby obtaining a cloned product;
(b) denaturing the cloned product, thereby obtaining a single-stranded polynucleotide template;
(c) hybridizing at least one mutagenized oligonucleotide primer to the single-stranded polynucleotide template, thereby obtaining a first heteroduplex; (
d) extending the first heteroduplex with a polymerase, thereby obtaining an extended product;
(e) reacting the extended product with a ligase, thereby obtaining ligated product;
(f) denaturing the ligated product, thereby obtaining a closed single stranded mutated polynucleotide;
(g) optionally repeating steps (c)-(f) (e.g., via thermal cycling);
(h) hybridizing the single stranded mutated polynucleotides with a second oligonucleotide primer thereby obtaining second hybridized complexes;
(i) copying the second hybridized complex and ligating the double stranded product thereof, thereby obtaining a circular double stranded mutated polynucleotide; and
(j) transforming the double-stranded mutated polynucleotide into a bacterial host, thereby obtaining transformants.

In one embodiment, the products can be subjected to optional PCR amplification, or DNA synthesis, such as about four or more cycles, to further increase the number of mutant products.

In another embodiment the invention provides for a kit for use in the methods described herein comprising:
(a) a vector comprising a cloning site;
(b) a generic oligonucleotide primer;
(c) a polymerase;
(d) a ligase;
(e) instructions for carrying out the method.

The invention also provides for primers and libraries of primers (e.g., two or more primers) for use in the claimed methods and methods of using mutagenized primers in the described methods.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 outlines the basic strategy used in INSULT, showing formation of multiple copies of closed mutant single stranded DNA in the first stage and binding of the generic primer to start the second stage. A single cycle of polymerase activity produces mutant closed circular homoduplex DNA; optional additional cycles PCR amplify the mutant product and linearly amplify one strand of parental DNA.

FIG. 2 is an agarose gel showing raw products of INSULT mutagenesis of small heat shock protein genes in two vectors. The intense single high molecular weight product in lane 7 in the transformant mutant (lower band is primers); lane 5 is similar except for the presence of weak artifact bands. Lane 2 contains an artifact at approximately equal strength to the product due to imperfect ligation. All these attempts were successful in producing the desired mutants without ultracompetent cells.

### DETAILED DESCRIPTION OF THE INVENTION

INSULT, a novel method for the creation of insertions, deletions, and point mutations without subcloning, requires only one new primer per mutant, and produces circular plasmids, obviating the need for special ‘ultracompetent’ cells. The method includes cycles of linear amplification with a thermophilic polymerase, and nick repair after each cycle with a thermophilic ligase. After production of multiple single stranded copies of circular mutation bearing plasmid DNA, addition of a ‘generic’ primer followed by one or more polymerase reaction cycles generates double stranded circular DNA bearing the desired mutation.

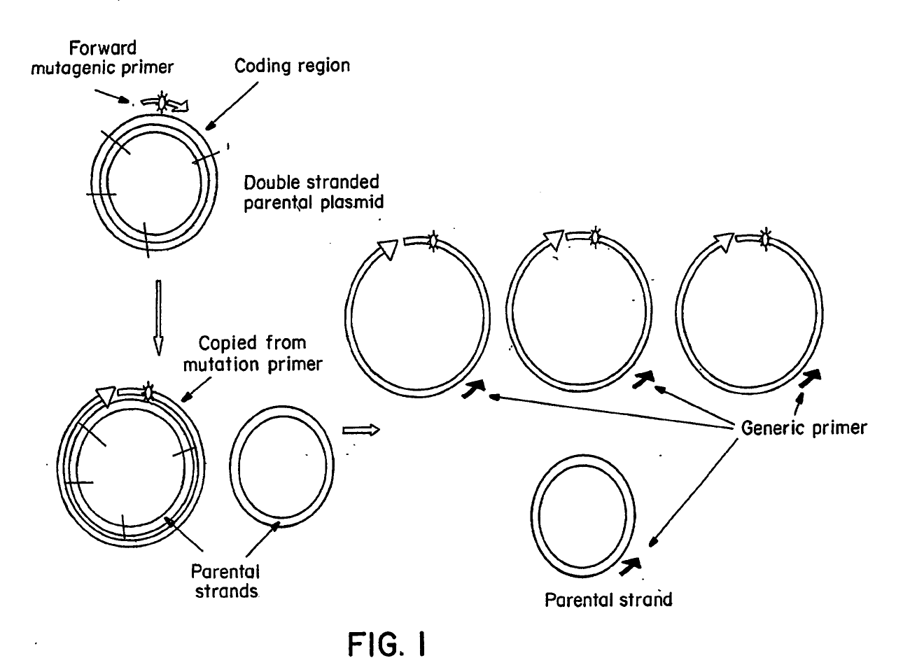

The basic strategy used in INSULT is outlined in FIG. 1. A single primer bearing a mutation is annealed to one strand of a denatured template consisting of double stranded closed circular plasmid carrying the gene (or other sequence) to be mutagenized. A polymerase, such as T4 or, preferably, thermophilic polymerase and thermophilic ligase (such as, Turbo pfu polymerase and Taq ligase), are added and the temperature cycled to produce single stranded closed circular copies of the target strand as described in the methods section. Use of a single primer produces linear amplification of the mutant strands. When T4 polymerase is employed, one preferably adds enzyme prior to or during each cycle to maximize activity. Thermophilic ligases can often be used without subsequently refreshing the reaction medium.

In one embodiment, the parental strand is destroyed in the reaction medium or selected against after transformation, for example, by using a selection primer, such as those provided with commercial kits, such as the Clontech Transformer Kit. Alternatively, the method is carried out in the absence of an oligonucleotide primer that repairs or inactivates a selection sequence.

The mutagenized oligonucleotide primer is capable of hybridizing to the polynucleotide sequence to be mutated and introduce one or more mutations. The primer can insert, delete or substitute/change one or more nucleotides (such as three or more nucleotides) or one or more codons (such as two, five or more codons), for example. Multiple primers (e.g., about 5, 10 or 20 or more) can be used that bind to the same, different, or overlapping or non-overlapping sequences of the parental polynucleotide. The preparation of mutagenizing primers is generally known in the art.

After production of a suitable number (e.g., preferably between about 10-20) of single stranded mutant copies, a ‘generic’ primer is introduced. This primer should not overlap the mutation, and it is desirable that no part of it be complementary to the mutagenizing primer. If many mutations to genes carried in a vector are contemplated, the generic primer can be made to a position in the vector outside the cloning site. If many mutations are to be made to a gene in different vectors, reverse or forward primers used for copying the gene, or internal sequencing primers which don't overlap the mutation primer, are suitable as long as the generic primer and the mutation primer anneal to opposite strands of the template.

In one embodiment, the mutagenized oligonucleotide primer further comprises a unique sequence (e.g. at least about 4 nucleotides) which hybridizes to the second oligonucleotide, or generic, primer, thereby introducing a simultaneous selection step in the DNA synthesis step.

Further adding a blocking oligonucleotide that hybridizes to the parental polynucleotide at or proximal to the sequences the mutagenized oligonucleotide primer hybridizes can additionally provide a negative selection for the parental polynucleotide.

One cycle of denaturation, annealing, and polymerase activity produces closed circular duplexes of the mutant and parentals; with the mutant DNA in great excess. Additional cycles pcr amplify the mutant DNA and linearly amplify one strand of the parental DNA. This leads to a huge excess of duplex mutant DNA, but many cycles of per could cause the accumulation of copy errors in the pool of mutants even with a high fidelity polymerase.

It is sometimes convenient to run the first stage overnight, and to finish the procedure with the short second stage (e.g. about 1-5 cycles) the next morning. This allow transformation and plating on selective media on the second day.

The process can be practiced conventiently with currently available vectors and thermophilic enzymes. Currently available kits, such as the Promega and Clontech mu genesis kits, can be adapted for use in the procedure, but the enzymes used in these kits are not thermostable. This limits them to a single thermal cycle per enzyme addition, which is not optimal. The vectors used can comprise an insertion site for introducing the parental polynucleotide. The vector can also further comprise a replication of origin, such as that of a filamentous bacteriophage, for example. The replication of origin is preferably an f1 replication origin.

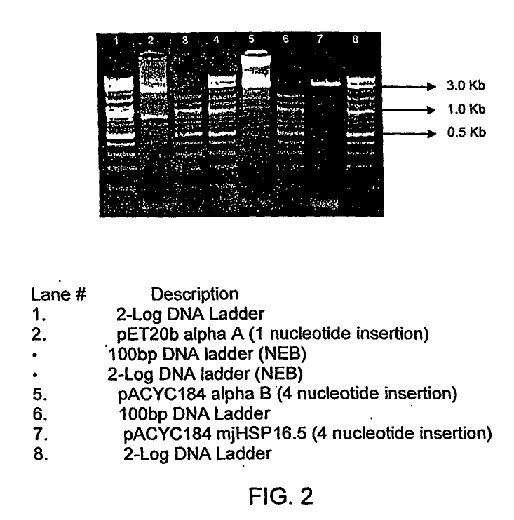

Initial experiments were designed to produce point mutants in the αA-crystalline pACYC184T7 system. The single mutation primers are shown in FIG. 2; the generic primers used in these experiments are simply the reverse primers that were originally used to copy the gene for introduction into the plasmid, and overlap the gene/plasmid junction. Any site separated from the mutation primer could have been used, although regions with moderate GC content are most efficient.

Transformation into BL21 cells with 1 uL of the reaction mixture produced about forty colonies on six plates, two for each mutant. As shown in table 1, the mutation frequency for the initial experiments was approximately 80%, and all three mutants were obtained on the first trial.

Production of insertion and deletion mutants was investigated using the same system (aA-crystallin pACYC184T7) with primers as indicated in Table 1. Results from these trials are summarized in Table 1. Insertions and deletions were obtained on the first attempt.

The eNOS pCWori+ system of approximately 9.5 kB represents a significant challenge for mutagenesis because of the presence of GC rich regions and recurring short motifs. Primers designed to insert a stop codon in the eNOS gene failed to produce any mutant colonies in several attempts with Stratagene QCM procedure or with our improved version using separate single primer linear amplification throughout, probably because of runaway PCR artifact.

Transformation of the same cell line (Stratagene XL10-Gold Ultracompetent cells) with the products of the mutagenesis procedure described here under the same conditions produced approximately 150 colonies per plate. As indicated in Table I, all of the colonies sampled were mutants, indicating that the mutation frequency is at least comparable to that obtained with the pACYC184T7 system. Direct comparison of these results suggests that for this mutant the new procedure is at least 1000 times more effective.

Selection Primer System. Several other selection systems are in use, including repair of antibiotic resistance genes and removal of restriction sites, which are features of the Promega and Clontech mutagenesis kits. The Promega kits was used to demonstrate the ability of the new procedure to use the selection protocol. The proprietary plasmid and repair primer generated colonies with the appropriate antibiotic resistance in the first attempt when transformed into Promega's competent cell line.

These results are significant because the new protocol transforms both the Promega and Clontech selection methods from a 50% theoretical mutation frequency to a 100% theoretical mutation frequency.

The production of mutant genes and their products without subcloning has been an important technical advance. Limitations of existing techniques flow naturally from flaws in strategy. QCM is well known to produce primer dimer in some situations, limiting its application in indel production. In addition, all QCM like procedures have the potential to degrade the mutant DNA they produce as the procedure is carried out. Although the mutant strands are never templates for the production of new mutant DNA from the mutagenic primers, the forward and reverse strands can prime each other for extension unless blocked by ‘wrong way’ (3′ out) primer binding. Where extension occurs, each strand is blunt ended, preventing the formation of circular DNA, and the gene is disrupted by the addition of a second copy of the primer sequence. To make matters worse, the duplexes destroyed by this process are now templates for runaway per, limiting the number of cycles of amplification that can be carried out. In favorable cases a high frequency of mutation can still be obtained, but the procedure still produces single stranded DNA requiring ultracompetent cells for transformation.

Clontech and Promega type strategies are limited by production of only a single copy of mutant DNA per parental, and by the production of hybrid duplexes which limits the selection power of antibiotic or restriction enzyme resistance. Production of high levels of mutant DNA is relatively easy by using thermostable enzymes that allow multiple copying steps. Introduction of a completely uncomplimentary, generic reverse primer makes INSULT qualitatively different from previous procedures, because the mutant copies produced are closed circular AND homoduplex. This is only possible because the multiple copies produced in stage 1 are in closed circular form; linear copies produced without ligase activity cannot be templates for synthesis of a reverse strand without introduction of primers to sites adjacent to the mutagenic primer, and this produces blunt ended linear duplexes.

Numerous variants of INSULT are feasible. Running a single cycle second stage decreases the amount of mutant DNA with the compensating advantage of introducing fewer copy errors. There are several options available for parental suppression. These include DPN1 digestion of methylated template as introduced by Strategene. Clontech and Promega selection strategies use a second forward ‘selection’ primer to repair an antibiotic resistance site or suppress a restriction site, and many other schemes are possible e.g., introduction of a mutation preventing induction of an inhibitory gene. These schemes are of real but limited utility in existing protocols because duplex DNA is a hybrid with one mutant and one parental strand, limiting selection efficiency to 50% with one transformation. Because INSULT produces homoduplexes, these selection schemes have a theoretical efficiency of 100% when applied within the INSULT context. This is true even in the limiting case when both the first and second stage are reduced to a single cycle, which would allow the use of T4 or other thermosensitive polymerase and ligase combinations. We believe that the T4 system is less desirable because of the lack of amplification of mutant DNA, but in view of the potential for total parental suppression this could be compensated for by increasing the level of template DNA.

The inherent ligase component of INSULT provides great potential for parallel introduction of multiple mutations. Multiple mutagenic primers would be extended by the polymerase to produce sections of DNA aligned along the circular template; the nicks separating the ends would be repaired by the ligase, generating multiple mutations in a single procedure. Limitations on this capability are imposed primarily by the need to not have the primers overlap, and in many cases closely spaced mutations could be carried on a single primer. Typically, the mutagenizing primers for point mutations are between about 15 and 35 basepairs (often 18-30 basepairs) in length. Mutations to two codons separate by less than half the primer length can most easily be accommodated by changing both codons in a single mutation. Mutagenizing primer design is generally known in the art. Combinatorial numbers of mutants and ‘limited chimera’ can in principle be constructed with a limited number of primers by applying the multiple mutation approach with mixtures of mutagenic primers. (The chimera produced are limited in scope by the size of the individual primers used). For example, n sets consisting of m mutagenic primers each, binding to n different sites within a gene, would generate mⁿ mutants from mⁿ primers when run together in the first stage. A single generic primer would suffice for the second stage. Use of a combinatorial mutagenic primer (a primer set in which all or many possible combinations of bases in a short stretch are present) would produce a combinatorial mixture of mutants concentrated in a single site. Since in all cases the mutants are produced without subcloning and transform directly into cell lines capable of expression, the system has great potential for selection-based applications.

A primary advantage of INSULT is the ability of the relatively high levels of circular duplex mutant DNA to transform expression competent cells directly. In most cases this represents a greater economy than the need for only one primer per mutation. More importantly, it removes the need for a second cycle of transformation to produce mutant proteins, which in most cases is the object of the exercise. This streamlining of the procedure greatly reduces the time and effort involved. In addition to saving human time, it moves the entire process into a form amenable to 96 well plates and robotics until the point of scale up from colony selection to protein production. In most cases expensive ‘Ultracompetent’ cells are unnecessary. On the other hand, the use of such cells in the INSULT process can produce very large numbers of mutants compared to other methods and allows the rapid production of mutants.

One skilled in the art will appreciate the many advantages that the method of the invention provides. For example, the improved site-directed mutagenesis methods of the invention are useful in protein and enzyme engineering technologies (to impart desirable properties on proteins, enzymes, polynucleotides, etc.) for the production of drugs, diagnostics, research proteins and enzymes, agrochemicals, plant proteins, industrial proteins and enzymes such as detergent enzymes, enzymes useful for neutralizing contaminants, and enzymes suitable for improved or novel biosynthesis of compounds in industry, biotechnology, and medicine. Likewise the methods of the invention are useful in protein engineering technologies for the production of proteins useful in the food and life sciences industries such as primary and secondary metabolites useful in the production of antibiotics, proteins and enzymes for the food industry (bread, beer), and combinatorial arrays of proteins for use in generating multiple epitopes for vaccine production. The invention can also be used to manufacture novel polynucleotides, including DNAs and RNAs, such as RNA inhibitors. In yet other embodiments, the inventions can be used to manufacture protein tags, such as N-terminal addressing, affinity tags, labeling sites, etc. The invention can be used in cell biology discovery and understanding protein-protein interactions. Fusion proteins for purification, targeting, labeling can be manufactured using the methods of the invention. For example, vectors with a GFP gene adjacent to a cloning site would allow easy conversion of a vector for expression of a target gene, e.g. via a linker.

### EXAMPLES

Methods

Polymerase and ligase reactions were carried out simultaneously in the same vessel. The reaction mixture consisted of 5 ul of 10× Reaction buffer, 10 ng of template DNA, 125 ng of phosphorylated mutagenesis primer, 5 ul 10 mM NAD+ (ligase cofactor), 1 ul 20 mM dNTP mix, 1 ul Pfu Turbo, 1 ul Taq DNA ligase, and dH20 addded to make the final reaction mixture 50 uL.

The thermocycler program consisted of two stages. In the first, the template was denatured at 94C for 2′, followed by annealing at 60C for 50 sec and extension for 10 minutes at 68C; on completion of extension around the plasmid the ligase closed the nicked product. Subsequent cycles (1-5) were identical except that the 94C step was shortened to 50 sec.

After holding at 4C, 2 ul 100 ng/ul phosphorylated universal primer was added to the reaction mixture in preparation for step 2. After denaturation at 94C for 2 minutes, the primers were annealed for 50 sec at 60C and extended at 68C, followed by nick repair. Up to four additional cycles followed as in the first stage.

50 uL of competent BL21DE3 cells were transformed with 1 uL reaction mixture, and the resulting transformed cells were plated on LB antimycin plates for selection of colonies. A representative fraction of antibiotic resistant colonies were selected and sequenced to confirm the production of mutants.

Transformation of the same cell line (Stratagene XL10-Gold Ultracompetent cells) with the products of the mutagenesis procedure described here under the same conditions produced approximately 150 colonies per plate. As indicated in Table I, all of the colonies sampled were mutants, indicating that the mutation frequency is at least comparable to that obtained with the pACYC184T7 system. Direct comparison of these results suggests that for this mutant the new procedure is at least 1000 times more effective.

TABLE 1

| | | Sequencing | |
|---|---|---|---|
| Clone name | Primer Sequence | Results | |
| Alpha A | F: CGC GAG TTC CAC GGC CGC TAC CGC CTG CCT TCC (SEQ ID NO. 1) | 2/3 | |
| Crystallin- | R: CC CCT CAA GAC CCG TTT AGA GGC CCC (SEQ ID NO. 2) | | |
| R116G | | | |
| Alpha A | F: GGA GAT ATA CAT ATG GGC ATC GCC ATT CAG CAC CCC TGG | (SEQ ID NO. 3) | 2/3 |
| Crystallin- | R: CC CCT CAA GAC CCG TTT AGA GGC CCC (SEQ ID NO. 4) | | |
| D2G | | | |
| Alpha A | F: GAG GTC CGA TCC GAC CGG AGC AAG TTT GTC ATC TTC CTG G (SEQ ID NO. 5) | 3/3 | |
| Crystallin- | R: CC CCT CAA GAC CCG TTT AGA GGC CCC (SEQ ID NO. 6) | | |
| D69S | | | |
| Alpha A | F: GCC CAG CTC TGC GCT GTG GAA G [symbol] CT CGA GCA CCA CCA CCA CC | 1/1 | |
| Crystallin- | (SEQ ID NO. 7) | | |
| ALWKG | R: CCC CCT CAA GAC CCG TTT AGA GGC CCC (SEQ ID NO. 8) | | |
| Chimera | F: CAG CTC TGC GCC [symbol] TC GTC CCT CGA GC (SEQ ID NO. 9) | 2/2 | |
| correction | R: CC CCT CAA GAC CCG TTT AGA GGC CCC | | |

Bold = substitution
Highlight = insertion

Forward (mutagenic) and reverse (generic) primers for initial trails with INSULT mutagenesis. Sequencing results indicate the number of correct mutant sequences and total trials.

FIG. 2 shows an agarose gel of the raw products of the INSULT process on three different small heat shock protein/vector combinations. Unlike any of the competing procedures, the transforming product is visible in all cases as a major band (usually the only major band apart from the primers). The production of large amounts of high quality (i.e., closed circular homoduplex) mutant DNA is a key to the success of the method.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for polynucleotide mutagenesis comprising:
(a) cloning a parental polynucleotide into a vector comprising a cloning site, thereby obtaining a cloned product;
(b) denaturing the cloned product, thereby obtaining a single-stranded polynucleotide template;
(c) hybridizing at least one mutagenized oligonucleotide primer to the single-stranded polynucleotide template, thereby obtaining a first heteroduplex;
(d) extending the first heteroduplex with a polymerase, thereby obtaining an extended product;
(e) reacting the extended product with a ligase, thereby obtaining ligated product;
(f) denaturing the ligated product, thereby obtaining a closed single stranded mutated polynucleotide;
(g) optionally repeating steps (c)-(f);
(h) hybridizing the single stranded mutated polynucleotides with a second oligonucleotide primer thereby obtaining second hybridized complexes;
(i) copying the second hybridized complex and ligating the double stranded product thereof, thereby obtaining a circular double stranded mutated polynucleotide; and
(j) transforming the double-stranded mutated polynucleotide into a bacterial host, thereby obtaining transformants.

2. The method of claim 1 wherein steps (c)-(f) are repeated at least about four times.

3. The method of claim 2 wherein the polymerase is a thermophilic polymerase.

4. The method of claim 1 wherein the ligase is Taq DNA ligase.

5. The method of claim 1 wherein steps (c)-(f) are repeated at least about 10 times.

6. The method of claim 5 wherein steps (c)-(f) are repeated between about 10 and 25 times.

7. The method of claim 1 wherein step (i) is subjected to a single cycle of DNA synthesis.

8. The method of claim 1 wherein step (i) is subjected to a less than about 10 cycles of DNA synthesis.

9. The method of claim 1 characterized by the absence of an oligonucleotide primer that repairs or inactivates a selection sequence.

10. The method of claim 1 further comprising the step of destroying the parental strand prior to amplification of the first heteroduplexes.

11. The method of claim 1 wherein the mutagenized oligonucleotide primer inserts one or more nucleotides into the parental polynucleotide.

12. The method of claim 1 wherein the mutagenized oligonucleotide primer inserts three or more nucleotides into the parental polynucleotide.

13. The method of claim 1 wherein the mutagenized oligonucleotide primer inserts two or more codons into the parental polynucleotide.

14. The method of claim 1 wherein the mutagenized oligonucleotide primer deletes one or more nucleotides in the parental polynucleotide.

15. The method of claim 1 wherein the mutagenized oligonucleotide primer deletes three or more nucleotides in the parental polynucleotide.

16. The method of claim 1 wherein the mutagenized oligonucleotide primer deletes two or more codons in the parental polynucleotide.

17. The method of claim 1 wherein the mutagenized oligonucleotide primer substitutes one or more nucleotides into the parental polynucleotide.

18. The method of claim 1 wherein the mutagenized oligonucleotide primer substitutes three or more nucleotides in the parental polynucleotide.

19. The method of claim 1 wherein the mutagenized oligonucleotide primer substitutes two or more codons into the parental polynucleotide.

20. The method of claim 1 wherein the mutagenized oligonucleotide primer substitutes five or more codons into the parental polynucleotide.

21. The method of claim 1 wherein two or more distinct mutagenized oligonucleotide primers are added in step (c).

22. The method of claim 1 wherein five or more distinct mutagenized oligonucleotide primers are added in step (c).

23. The method of claim 22 wherein each distinct mutagenized oligonucleotide primer substitutes two or more codons into the parental polynucleotide.

24. The method of claim 22 wherein each mutagenized oligonucleotide primer substitutes five or more codons into the parental polynucleotide.

25. The method of claim 1 wherein the parental polynucleotide comprises a coding sequence.

26. The method of claim 1 wherein the second oligonucleotide primer is not the complement of the mutagenized oligonucleotide primer or overlapped with it.

27. The method of claim 26 wherein the second oligonucleotide primer does not hybridize to the parental polynucleotide.

28. The method of claim 26 wherein the second oligonucleotide primer hybridizes to a sequence of the vector.

29. The method of claim 1 wherein the vector further comprises a replication origin of a filamentous bacteriophage.

30. The method of claim 29 wherein the replication origin is an f1 replication origin.

31. The method of claim 1 wherein in step (c), 5 or more mutagenized oligonucleotide primers are added to the single-stranded polynucleotide template.

32. The method of claim 31 wherein the 5 or more mutagenized oligonucleotides hybridize to substantially the same sequences on the single-stranded polynucleotide template.

33. The method of claim 31 wherein the 5 or more mutagenized oligonucleotides hybridize to different sequences on the single-stranded polynucleotide template.

34. The method of claim 33 wherein the 5 or more mutagenized oligonucleotides hybridize to non-overlapping sequences on the single-stranded polynucleotide template.

35. The method of claim 1 wherein in step (c), 10 or more mutagenized oligonucleotide primers are added to the single-stranded polynucleotide template.

36. The method of claim 1 wherein in step (c), 20 or more mutagenized oligonucleotide primers are added to the single-stranded polynucleotide template.

37. The method of claim 1 wherein the mutagenized oligonucleotide primer further comprises a unique sequence which hybridizes to the second oligonucleotide primer.

38. The method of claim 37 wherein the unique sequence is at least about 4 nucleotides.

39. The method of claim 38 further comprising the step of adding a blocking oligonucleotide that hybridizes to the parental polynucleotide at or proximal to the sequences the mutagenized oligonucleotide primer hybridizes, thereby providing a negative selection for the parental polynucleotide.

40. A kit for use in the method of claim 1 comprising:
(a) a vector comprising a cloning site;
(b) a generic oligonucleotide primer;
(c) a polymerase;
(d) a ligase;
(e) instructions for carrying out the method.

41. A method of using a kit comprising:
(a) a vector comprising a cloning site;
(b) a generic oligonucleotide primer;
(c) a polymerase;
(d) a ligase; and
(e) instructions for carrying out the method, in a method comprising the steps of:
(a) cloning a parental polynucleotide into a vector comprising a cloning site, thereby obtaining a cloned product;
(b) denaturing the cloned product, thereby obtaining a single-stranded polynucleotide template;
(c) hybridizing at least one mutagenized oligonucleotide primer to the single-stranded polynucleotide template, thereby obtaining one or more first heteroduplexes;
(d) subjecting the first heteroduplexes to linear amplification, thereby obtaining amplified products;
(e) reacting the amplified products with a ligase, thereby obtaining ligated products;
(f) denaturing the ligated products, thereby obtaining single stranded mutated polynucleotides;
(g) hybridizing the single stranded mutated polynucleotides with a second oligonucleotide primer thereby obtaining second hybridized complexes;
(h) subjecting the second hybridized complex to at least one cycle of DNA synthesis and ligating the double stranded products thereof, thereby obtaining a circular double stranded mutated polynucleotide;
(i) transforming the double-stranded mutated polynucleotide into a bacterial host, thereby obtaining transformants.

42. A library comprising two or more mutagenized oligonucleotide primers for use in the method of claim 1.

43. A method of using a library comprising two or more mutagenized oligonucleotide primers comprising the steps of:
(a) cloning a parental polynucleotide into a vector comprising a cloning site, thereby obtaining a cloned product;
(b) denaturing the cloned product, thereby obtaining a single-stranded polynucleotide template;
(c) hybridizing at least one mutagenized oligonucleotide primer to the single-stranded polynucleotide template, thereby obtaining one or more first heteroduplexes;
(d) subjecting the first heteroduplexes to linear amplification, thereby obtaining amplified products;
(e) reacting the amplified products with a ligase, thereby obtaining ligated products;
(f) denaturing the ligated products, thereby obtaining single stranded mutated polynucleotides;
(g) hybridizing the single stranded mutated polynucleotides with a second oligonucleotide primer thereby obtaining second hybridized complexes;
(h) subjecting the second hybridized complex to at least one cycle of DNA synthesis and ligating the double stranded products thereof, thereby obtaining a circular double stranded mutated polynucleotide;
(i) transforming the double-stranded mutated polynucleotide into a bacterial host, thereby obtaining transformants.
